(19) United States
(12) Patent Application Publication
(10) Pub. No.: US 2009/0266728 A1
(43) Pub. Date: Oct. 29, 2009

(54) MEDICAL DEVICE TRACKING SYSTEM WITH TRAY AND METHOD

(75) Inventors: Robin L. TURNER, Memphis, TN (US); Sanjay BISHT, Memphis, TN (US); Thor M. HANNA, JR., Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/109,539

(22) Filed: Apr. 25, 2008

(51) Int. Cl. A61B 19/02

(52) U.S. Cl. 206/363; 206/459.5; 206/459.1

(57) ABSTRACT

Embodiments of the invention include systems and methods for tracking a medical device, including tracking medical devices in a set or tray. Systems configured for such tracking may include the capability to either or both detect tampering with the medical device and to effectively expose one or more medical devices to sterilization substances.

### FIELD OF THE INVENTION

The present invention relates generally to the field of identification and tracking of parts, and more particularly relates to tracking a medical device by associating the medical device with identifying information and providing the medical device in a tray.

### BACKGROUND

Implantable medical devices must be sterile prior to use in order to reduce the risk of infection in patients receiving such devices. Generally, there are two ways to provide sterile surgical devices. One way is to sterilize a device to be implanted immediately prior to implantation. Another way is to sterilize a device during the manufacturing process, and then to ship the device to a user in a sterilized condition. The first way is typically called providing a device “non-sterile,” because the manufacturer ships the device in a condition that is not adequately sterilized for implantation. The second way is typically called providing a device “sterile,” because the device is ready for implantation when shipped from the manufacturer.

There is a strong and growing need to track medical devices from their base materials and manufacture to their use, and throughout the intervening time. Tracking of medical devices may also be referred to as maintaining traceability of the devices. It is sometimes important to track medical devices so that patients can be notified of any information related to the safety or longevity of devices once implanted. The U.S. Food and Drug Administration is currently considering requiring that implantable medical devices be uniquely identified and tracked through the time of use of the devices.

It is relatively straightforward to uniquely identify and track sterile medical devices. Unique labels or other indicia are applied to the product and the labels or other indicia remain associated with the medical device until the device is used. In some instances, sterile product labels include adhesive portions that can be applied to a chart or file of a patient to conveniently associate the sterile medical device with a particular patient.

Non-sterile products provide a greater tracking challenge, although there are several reasons for preferring non-sterile shipment of medical devices. A larger number of non-sterile devices can be provided in groups or sets that present the devices in a manner where the devices are readily available for use. The large number of devices may represent a large number of sizes and optional configurations that provide surgeons with many alternatives in a convenient arrangement. Devices that are not used are simply returned to stock for sterilization prior to a subsequent use. Non-sterile devices do not have a definitively limited shelf life, as sterile products do. Non-sterile devices are less expensive to package and sterilize. Non-sterile devices can typically be more densely packaged into a common carrier than sterile devices. The primary reason that such non-sterile products are difficult to track, however, is that the products are difficult to mark, may not be marked at all, and may be identical to other products with which they are packaged, thus creating a possibility of confusion among parts. In many instances, specific non-sterile products are not tracked beyond their manufacturing facility, and may only be counted when reconciled for payment as one of many products that were not returned to a manufacturer for replenishment.

One way of tracking non-sterile medical devices would be to associate the devices with a mechanism that includes identifying information. The mechanisms may be provided in multiples in, for instance, trays. Such mechanisms may provide ready access to the device by sterilizing material such as steam or other cleaning solutions. It would be advantageous in some tracking systems for non-sterile implants to be resistant to intentional or even incidental tampering that could disassociate identifying information from a medical device.

### SUMMARY

An embodiment of the invention is a medical device tracking system. The system includes a tray, a medical device carried by the tray, and a mechanism coupled to the tray. The mechanism is associated with the medical device and the mechanism includes a distal portion configured to couple with the tray and an identifier associated with the mechanism. In some embodiments, the mechanism captures the medical device relative to the tray and is configured such that removal of the medical device from at least a portion of the mechanism is thereafter detectable.

Another embodiment of the invention is a medical device tracking system that includes a tray, multiple medical devices carried by the tray, and multiple mechanisms coupled to the tray, the mechanisms being respectively associated with the multiple medical devices. One or more of the mechanisms has a distal portion configured to couple with the tray in a location on the tray that is associated by proximity with the medical device, and an identifier associated with the mechanism. The multiple mechanisms of some embodiments do not contact the respective multiple medical devices.

An embodiment of the invention is a method of tracking medical devices delivered in a set that includes multiple medical devices. The method comprises providing a tray, providing two or more medical devices in the tray, and providing identifiers associated with respective ones of the medical devices. The method may also include giving instructions to detach one or more identifiers to record which medical devices have been used. The act of detaching one or more identifiers may thereafter be detectable.

Yet another embodiment of the invention is a method of tracking multiple medical devices includes providing a tray configured to accept multiple medical devices and respective multiple identifiers, and providing instructions to sterilize the medical devices carried in the tray. The method may also include receiving the tray after one or more of the multiple medical devices has been removed, but with one or more associated identifiers retained and recording the retained identifiers to at least track which medical devices have been used.

### BRIEF DESCRIPTION OF THE DRAWINGS

FIG. 1 is a perspective view of an embodiment of the invention that illustrates a tracking system including a spinal pedicle screw.

FIG. 2 is a plan view of the embodiment of FIG. 1.

FIG. 3 is an elevation view of the embodiment of FIG. 1.

FIG. 4 is a perspective view of an embodiment of the invention that illustrates a tracking system including a spinal pedicle screw.

FIG. 5 is a perspective view of an embodiment of the invention that illustrates a tracking system including a medical device in a container.

FIG. 6 is s perspective view of components of FIG. 5 with the medical device and container removed for clarity.

FIG. 7 is a perspective view of an embodiment of the invention that illustrates a tracking system including spinal pedicle screws in a tray.

FIG. 8 is an elevation view of an embodiment of the invention that illustrates at least a portion of a tracking system including a spinal pedicle screw.

FIG. 9 is a perspective view of an embodiment of the invention that illustrates a tracking system including spinal pedicle screws in a tray.

FIG. 10 is a flowchart directed to method embodiments of the invention.

FIG. 11 is a flowchart directed to method embodiments of the invention.

### DETAILED DESCRIPTION

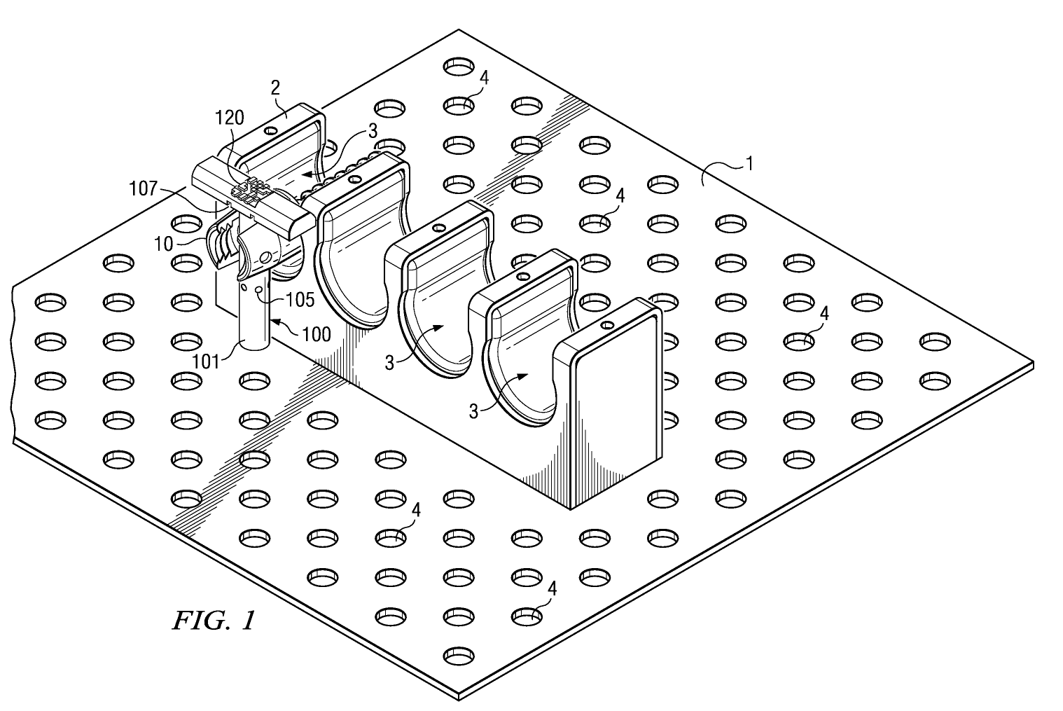

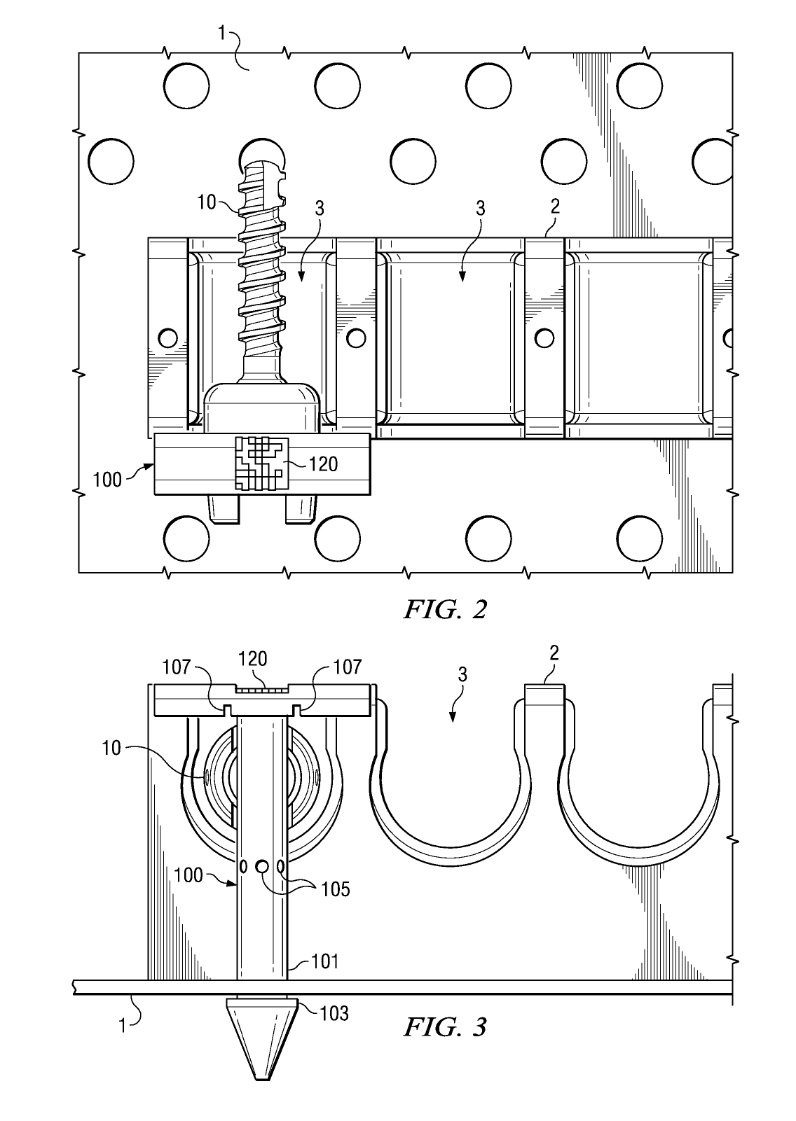

FIGS. 1-3 illustrate an embodiment of a medical device tracking system. As illustrated, a tray 1 includes a carrier 2. The tray 1 shown is a planar segment with multiple holes 4. The holes 4 may be useful for permitting steam or another sterilizing substance to enter the tray 1 and contact a medical device carried by the tray, such as the spinal surgical screw 10. Sterilization may be from steam or from application of a chemical sterilizing substance, or from any other effective sterilization substance or process. The tray may be a single planar carrier as illustrated, or may contain one or more of a side or sides and a full or partial lid. As shown, the carrier 2 includes multiple bays 3 capable of receiving medical devices, even though only one on the bays is occupied in FIGS. 1-3.

A medical device, such as spinal surgical screw 10 may be placed in the carrier 2 of the tray 1. As shown, a spinal surgical screw 10 is placed in a bay 3 of the carrier 2 of the tray 1. The medical device of this or any other embodiment of the invention may be any implant or instrument used in accomplishing a medical procedure. The medical device of some embodiments is capable of undergoing one or more steam sterilization cycles, or other sterilization procedures, without degrading in a manner that would make the implant unsuitable for use in a medical procedure. The medical device of this or any other embodiment of the invention may consist of materials, by way of example, and without limitation, including titanium and its alloys, ASTM material, cobalt chrome, tantalum, ceramic, poly-ether-ether-ketone (PEEK), PEAK, various plastics, plastic composites, carbon fiber composites, coral, allograft, autograft, zenograft, and can include artificial materials which are at least in part bioresorbable, or any material suitable for human implantation.

In addition to being a spinal surgical screw, the medical device may be, without limitation, a surgical screw of any variety, a spinal or other orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, or other spinal fixation instrumentation, an intramedullary nail, an external fixation device, a hip prosthesis or therapeutic device, a knee prosthesis or therapeutic device, or an instrument useful with any of the previously recited devices.

As illustrated in FIGS. 1-3, a mechanism 100 is coupled to the tray 1. The mechanism 100 depicted is associated with the spinal surgical screw 10 by both contact and proximity. The mechanism 100 shown has a distal portion 101 configured to couple with the tray 1, and as particularly illustrated, the distal portion 101 couples into a hole 4 in the tray 1. In other embodiments, the mechanism 100 may couple, without limitation, with a side, bottom, lid, separator, or other feature of a tray. The distal portion 101, shown more clearly in FIG. 3, couples into the hole 4 by snapping into or being pushed through the hole 4. Once through the hole 4, a shoulder 103 at the distal portion 101 prevents the distal portion 101 from being easily removed from the tray 1. In other embodiments, the distal end 101 of the mechanism 100 may be coupled to the tray 1, without limitation, by a threaded connection, a transverse fastener, a pin, a clip, an adhesive, by melting, by welding, or by any other effective way of coupling. The part of the mechanism 100 describe as the distal portion 101 herein is described as “distal” as merely a relative reference and is not required to be opposite from any particular feature of the mechanism, tray, or medical device in various embodiments of the invention.

As illustrated in FIGS. 1-3, an identifier 120 is associated with the mechanism 100. The illustrated identifier 120 includes a two dimensional bar code. The identifier 120 may be any device that is capable of retaining identifying information. In some embodiments, the identifier 120 is a device suitable for scanning by an optical scanner such as a one or two dimensional bar code reader. The identifier 120 may also be a radio frequency identification (RFID) device that is readable through radio frequency transmission generated by an independently powered RFID device. The identifier 120 may be an RFID device that includes a transponder and is readable in response to a radio frequency signal transmitted to the RFID device. In some embodiments, the identifier 120 is a human readable visual and/or tactile device such as, but not limited to, alphanumeric characters, and may optionally include raised or lowered portions.

In some embodiments, the mechanism 100 captures the medical device relative to the tray 1 and is configured such that removal of the medical device from the mechanism 100 is thereafter detectable. As shown in FIGS. 1-3, the mechanism 100 captures the spinal surgical screw 10 relative to the tray 1 by securing the distal end 101 of the mechanism 100 into the tray and trapping the spinal surgical screw 10 between the carrier 2 of the tray 1 and the mechanism 100. Once trapped in this position, the mechanism 100 or tray 1 must be altered in some way to thereafter remove the spinal surgical screw 10. Alteration to the mechanism 100 of the embodiment illustrated in FIGS. 1-3 is facilitated by a structurally weakened portion 105 (FIGS. 1 and 3) of the mechanism 100. The illustrated structurally weakened portion 105 includes openings that extend through the mechanism 100. Additional or alternate structurally weakened portions 107 (FIGS. 1 and 3) of the mechanism 100 are also shown. The alternate structurally weakened portions 107 are grooves that reduce, for example, the bending strength of the mechanism 100 at the location of the grooves.

In operation, force applied to the mechanism 100 directly, or as a result of force applied through the spinal surgical screw 10 may apply forces to the structurally weakened portion 105 or alternate structurally weakened portions 107. In some embodiments, the structurally weakened portion 105 or alternate structurally weakened portions 107 will rupture, non-resiliently deform, or change color prior to any other portion of the mechanism 100, and the spinal surgical screw 10 will then be removable from the tray 1 and at least a portion from the mechanism 100. A rupture can be a full or partial rupture. Likewise, a non-resilient deformation may be a change in the shape that remains thereafter and is in whole or in part detectable. A discoloration may occur, for example and without limitation, when a polymer material undergoes plastic material deformation. In other embodiments, the detectable alteration may be to any part of the mechanism 100 or the tray 1, or any subcomponent or additional component added to the system. Rupture, non-resilient deformation, change in color, or any other detectable indicia may be evident from observation of any part of the components or subcomponents of an embodiment of the system, and not just evident from alteration to a structurally weakened area.

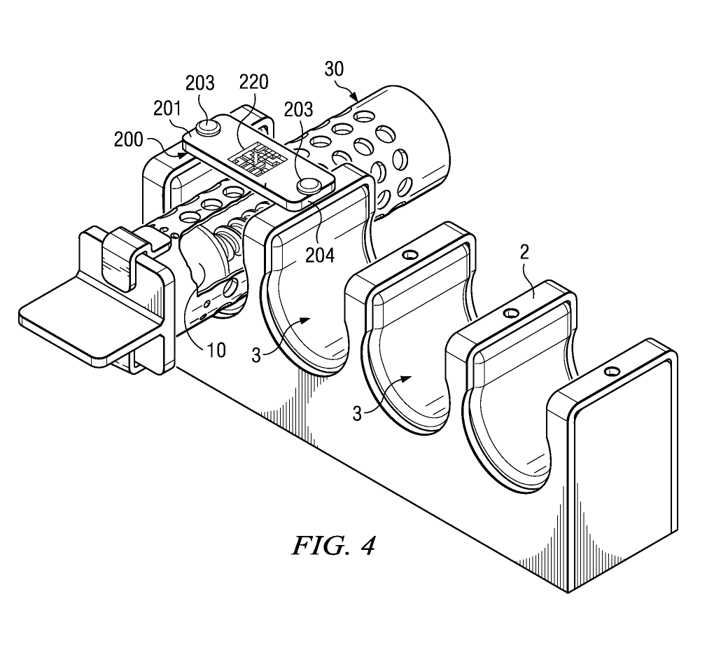

FIG. 4 illustrates an embodiment of a medical device tracking system. A carrier 2, as described more fully above, is shown. The carrier 2 may be part of a tray as described in association with FIG. 1-3. A medical device, such as spinal surgical screw 10 may be placed in the carrier 2. As shown, a spinal surgical screw 10 is contained within a capsule 30 and placed in a bay 3 of the carrier 2. The medical device of this or any other embodiment of the invention may be any implant or instrument used in accomplishing a medical procedure. The medical device may be, without limitation, similar to any of the medical devices described in association with FIGS. 1-3.

An identifier 220 is associated with a mechanism 200. The illustrated identifier 220 includes a two dimensional bar code. The identifier 220 may be any device that is capable of retaining identifying information, including but not limited to, all types described in association with the identifier 120 described herein.

In some embodiments, the mechanism 200 captures the medical device relative to the carrier 2 portion of a tray and is configured such that removal of the medical device from the mechanism 200 is thereafter detectable. As shown in FIG. 4, the mechanism 200 captures the spinal surgical screw 10 relative to the tray 1 by securing the capsule 30 in which the spinal surgical screw 10 resides within a bay 3 of the carrier 2. In other embodiments, a medical device may be directly captured relative to the carrier 2. Once trapped in this position, the mechanism 200, carrier, or connection between them must be altered in some way to thereafter remove the spinal surgical screw 10. Similarly, the capsule 30 may be altered to remove the spinal surgical screw 10. In some embodiments, the capsule 30 is restricted from moving longitudinally, thus requiring removal through the mechanism 200.

In operation, force applied to the mechanism 200 directly, or as a result of force applied through the spinal surgical screw 10 or capsule 30 may result in the rupture, non-resilient deformation, or change in color of the mechanism 200 or a connection between the mechanism 200 and the carrier 2. The mechanism 200 illustrated includes a first end 201 and a second end 204. Each of the first and second ends 201, 204 shown is coupled to the carrier 2 by a fastener 203. A rupture can be a full or partial rupture of, for example and without limitation, the first end 201, the second end 204, the body of the mechanism 200, or one or both of the fasteners 203. Likewise, a non-resilient deformation may be a change in the shape that remains thereafter and is in whole or in part detectable. A discoloration may occur, for example and without limitation, when a polymer material undergoes plastic material deformation. In other embodiments, the detectable alteration may be to any part of the mechanism 200 or the carrier 2 or other part of a tray, or any subcomponent or additional component added to the system. Rupture, non-resilient deformation, change in color, or any other detectable indicia may be evident from observation of any part of the components or subcomponents of an embodiment of the system.

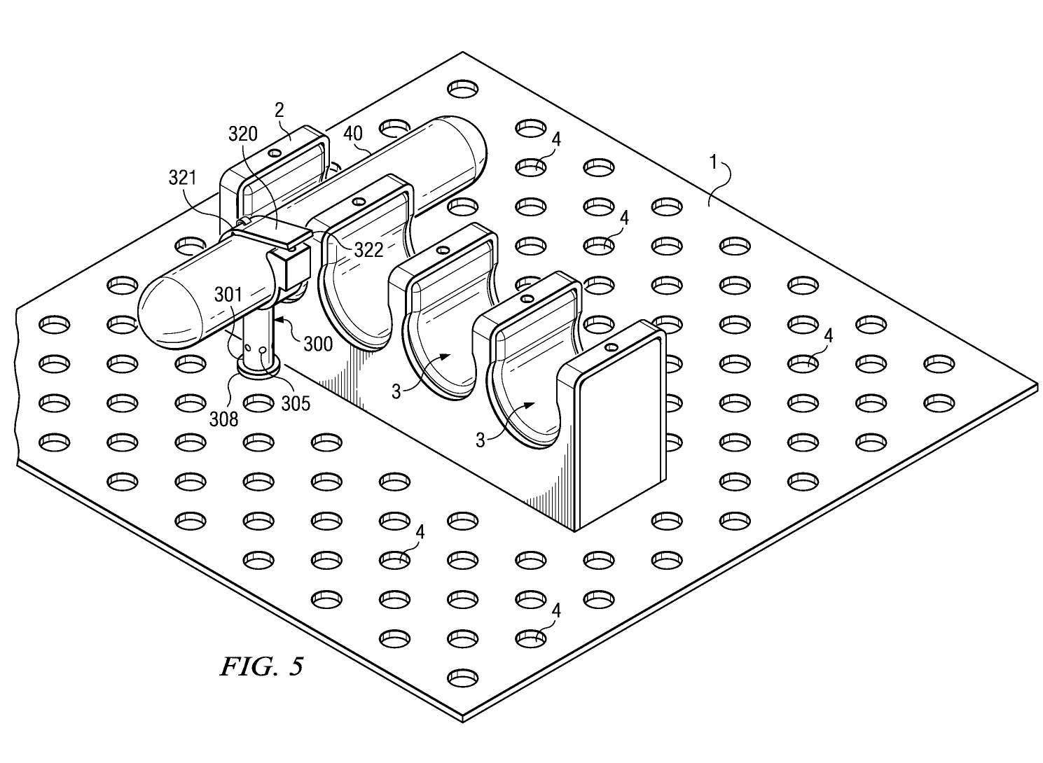

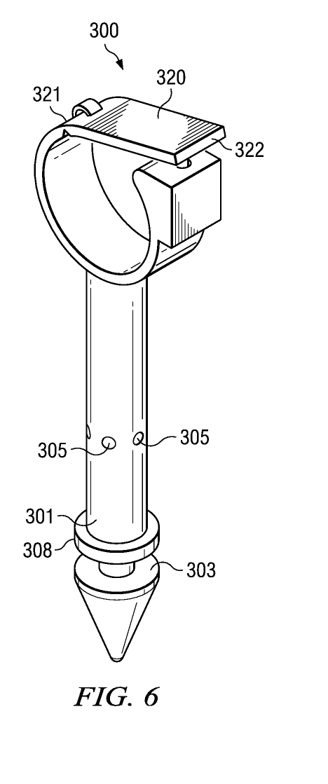

FIGS. 5 and 6 illustrate an embodiment of a medical device tracking system. As illustrated, a tray 1 includes a carrier 2. The tray 1 shown is a planar segment with multiple holes 4, as further described in association with FIGS. 1-3 herein. A medical device, such as spinal surgical screw in a capsule 40, may be placed in the carrier 2. In other embodiments, a medical device may be placed directly in the carrier 2 and not be carried in a capsule. As shown, the capsule 40 is placed in a bay 3 of the carrier 2. The capsule 40 of some embodiments is a hermetically sealed enclosure for the medical device. Therefore, in some embodiments, a medical device may be sterilized to meet a specific sterilization technique, such as gamma irradiation, and separately sealed in a steam sterilizable capsule that closely conforms to the size and shape of a medical device. With such an embodiment, multiple encapsulated medical devices may be provided more easily in a set and otherwise handled with the advantages of non-sterile medical devices.

A mechanism 300 in the illustrated embodiment is coupled to the tray 1. The mechanism 300 depicted is associated with a medical device within the capsule 40 by both contact and proximity to the capsule 40. The mechanism 300 shown has a distal portion 301 configured to couple with the tray 1, and as particularly illustrated, the distal portion 301 couples into a hole 4 in the tray 1. In other embodiments, the mechanism 300 may couple, without limitation, with a side, bottom, lid, separator, or other feature of a tray. The distal portion 301, shown more clearly in FIG. 6, couples into the hole 4 by snapping into or being pushed through the hole 4. Once through the hole 4, a shoulder 303 at the distal portion 301 prevents the distal portion 301 from being easily removed from the tray 1. The illustrated embodiment also includes a ring 308 that prevents the mechanism 300 from penetrating into the tray 1 further than desired. In other embodiments, the distal end 301 of the mechanism 300 may be coupled to the tray 1, without limitation, by a threaded connection, a transverse fastener, a pin, a clip, an adhesive, by melting, by welding, or by any other effective way of coupling. The part of the mechanism 300 describe as the distal portion 301 herein is described as “distal” as merely a relative reference and is not required to be opposite from any particular feature of the mechanism, tray, or medical device in various embodiments of the invention.

The medical device of this or any other embodiment of the invention may be any implant or instrument used in accomplishing a medical procedure. The medical device may be, without limitation, similar to any of the medical devices described in association with FIGS. 1-3.

An identifier 320 is associated with the mechanism 300. The identifier 320 may include a two dimensional bar code, an RFID tag, or any device that is capable of retaining identifying information, including but not limited to, devices described in association with the identifier 120 herein. The identifier 320 that is shown has a proximal end 321 and a distal end 322. The proximal end 321 illustrated is hinged and the distal end 322 is connectable to capture the capsule 40 in the mechanism 300. The mechanism 300 of this and other embodiments captures the medical device relative to the tray 1 and is configured such that removal of the medical device from the mechanism 300 is thereafter detectable. Once captured in this position, the mechanism 300 or tray 1 must be altered in some way to thereafter remove the capsule 40 and medical device. Alteration to the mechanism 300 of the embodiment illustrated in FIGS. 5 and 6 may be facilitated by a structurally weakened portion 305 of the mechanism 300. The illustrated structurally weakened portion 305 includes openings that extend through the mechanism 300.

In operation, force applied to the mechanism 300 directly, or as a result of force applied through the capsule 40 may apply forces to the structurally weakened portion 305. In some embodiments, the structurally weakened portion 305 will rupture, non-resiliently deform, or change color prior to any other portion of the mechanism 300, and the capsule 40 containing the medical device will then be removable from the tray 1. A rupture can be a full or partial rupture. Likewise, a non-resilient deformation may be a change in the shape that remains thereafter and is in whole or in part detectable. A discoloration may occur, for example and without limitation, when a polymer material undergoes plastic material deformation. In other embodiments, the detectable alteration may be to any part of the mechanism 300 or the tray 1, or any subcomponent or additional component added to the system. Rupture, non-resilient deformation, change in color, or any other detectable indicia may be evident from observation of any part of the components or subcomponents of an embodiment of the system, and not just evident from alteration to a structurally weakened area. For example, the proximal end 321 or the distal end 322 or any portion of the identifier 320 may manifest alteration of the mechanism 300 due to removal of the medical device from the mechanism.

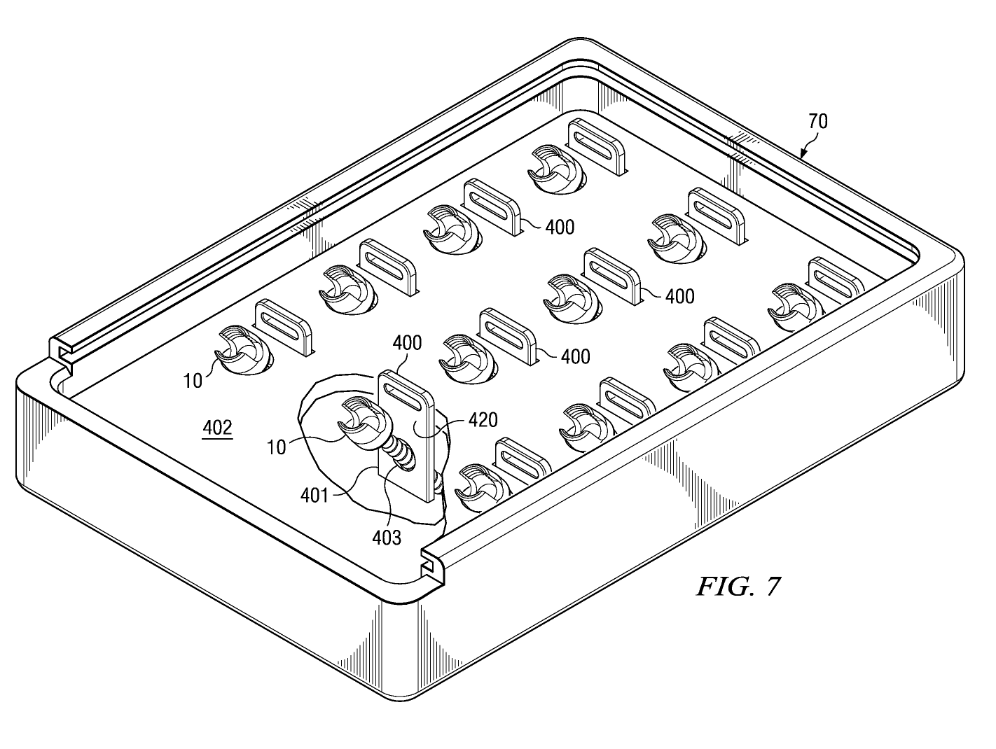

FIG. 7 shows a medical device tracking system with a tray 70, multiple medical devices, such as spinal surgical screws 10 carried by the tray 70, and multiple mechanisms 400 coupled to the tray 70. The tray 70 may be referred to as a caddy, container, or by other descriptive terms in some embodiments. The medical devices of certain embodiments may be any implant or instrument used in accomplishing a medical procedure. The medical device may be, without limitation, similar to any of the medical devices described in association with FIGS. 1-3. The mechanisms 400 illustrated are respectively associated with the spinal surgical screws 10, at least by proximity. The mechanisms 400 of some embodiments include a distal portion 401 configured to couple with the tray 70. Each mechanism 400 illustrated is in a location on the tray 70 that is associated by proximity with a respective spinal surgical screw 10. An identifier 420 may be associated with one or more of the mechanisms 400. The identifier 420 may include a two dimensional bar code, RFID tag, or any device that is capable of retaining identifying information, including but not limited to, devices described in association with the identifier 120 herein.

In some embodiments, the multiple mechanisms 400 do not contact the respective multiple medical devices. Instead, respective medical devices may be associated with mechanisms 400 by proximity. In other embodiments, there may be contact and interdigitation between respective mechanisms 400 and medical devices, such as spinal surgical screws 10. In some embodiments, a mechanism 400 may not be releasable from the tray 70 unless the medical device is first removed. For example, and without limitation, the system of FIG. 7, may include a mechanism 400 with a hole through which a respective spinal surgical screw 10 is inserted below the upper surface 402. The mechanism 400 and the spinal surgical screw 10 may be inserted along converging paths so that the spinal surgical screw 10 intersects a hole 403 in the mechanism 400. Removing the spinal surgical screw 10 may then release the mechanism 400 to either be removed from the tray 70, or to fall into a collection volume below the upper surface 402. This release is another example of a detectable indicator of removal of a medical device.

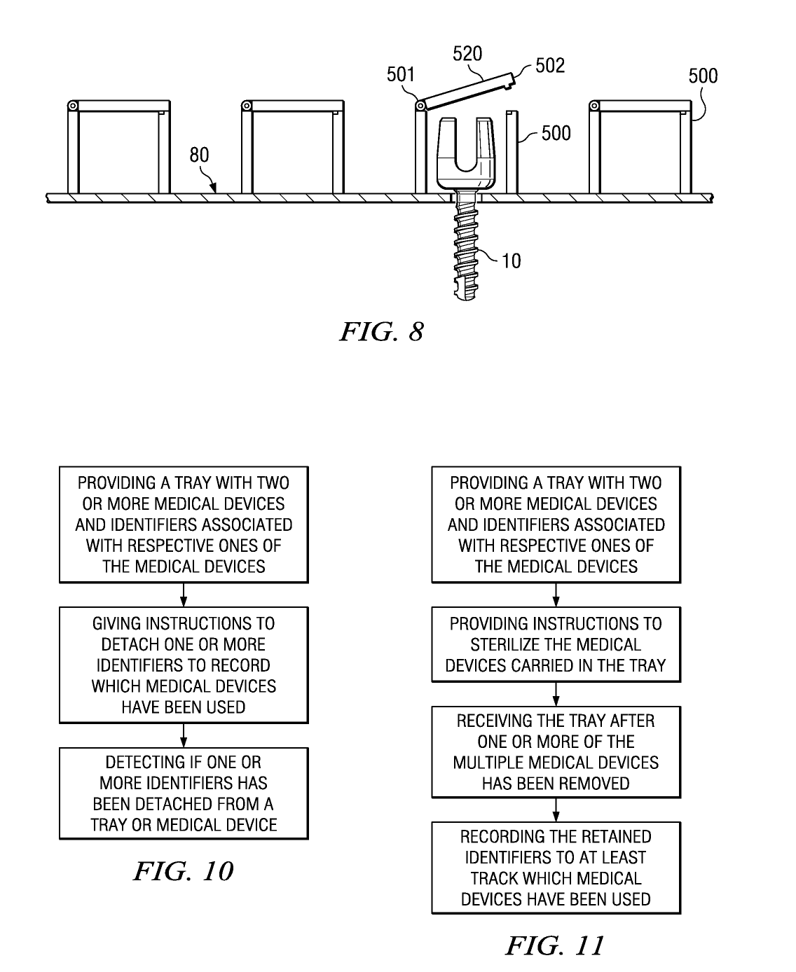

FIG. 8 shows a medical device tracking system with a tray 80, multiple medical devices, such as spinal surgical screws 10 carried by the tray 80, and multiple mechanisms 500 coupled to the tray 80. The tray 80 may be referred to as a caddy, container, or by other descriptive terms in some embodiments. The medical devices of certain embodiments may be any implant or instrument used in accomplishing a medical procedure. The medical device may be, without limitation, similar to any of the medical devices described in association with FIGS. 1-3. The mechanisms 500 illustrated are respectively associated with the spinal surgical screws 10, at least by proximity. The mechanisms 500 of some embodiments includes a distal portion 501 configured to couple with the tray 80, or another portion of the mechanism 500 that in turn connects with the tray 80. In FIG. 8, the distal portion 501 is coupled with the tray 80 by connection to mechanism 500 through a hinged coupling. A proximal portion 502 of the mechanism 500 may be detachably engagable with a latch on an adjacent portion of the mechanism 500 to capture the spinal surgical screw 10 in the tray 80. In other embodiments, a spinal surgical screw 10 or other medical device may be captured with a tray 80 by, without limitation, a sliding, treaded, snap fit, twist-off, pinned, clipping, or other effective device. Each mechanism 500 illustrated is in a location on the tray 80 that is associated by proximity and contact with a respective spinal surgical screw 10.

An identifier 520 may be associated with one or more of the mechanisms 500. As illustrated, the identifier 520 is incorporated into a hinged lid having proximal portion 502 and distal portion 501 that is coupled with the tray 80. The identifier 520 may include a two dimensional bar code, RFID tag, or any device that is capable of retaining identifying information, including but not limited to, devices described in association with the identifier 120 herein. In some embodiments, the identifier 520 is removable from the tray 80. For example, hinged distal portion 501 may be weakened along the hinged connection such that the identifier may be readily grasped and pulled or twisted from the tray 80. The mechanism 500 may be any functional cross-sectional shape such as, and without limitation, generally circular, oval, square, triangular, rectangular or any partial or combination shape that effectively contains a medical device or portion of a medical device.

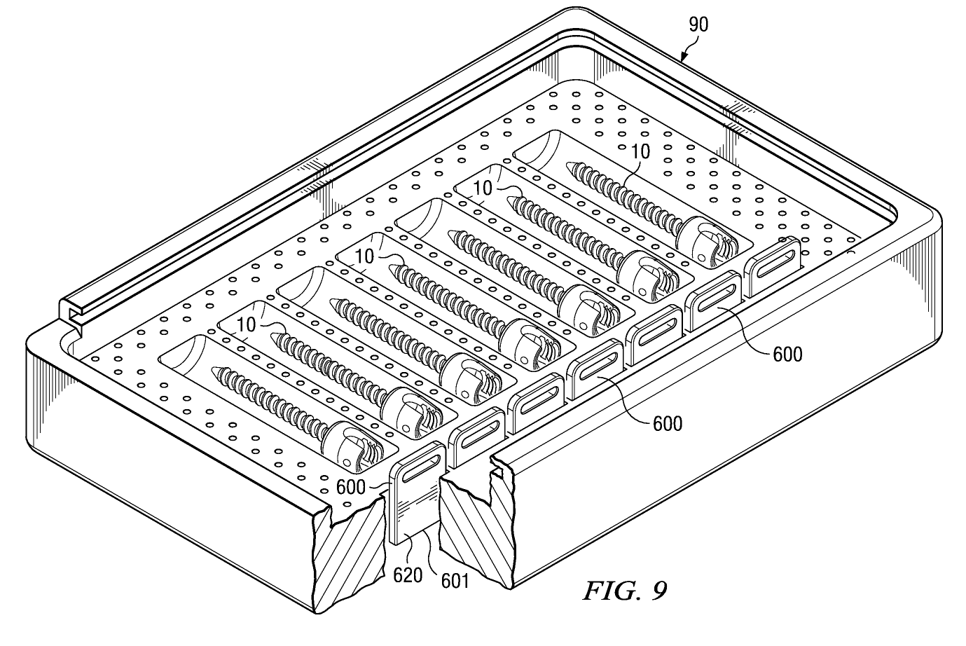

FIG. 9 illustrates a medical device tracking system with a tray 90, multiple medical devices, such as spinal surgical screws 10 carried by the tray 90, and multiple mechanisms 600 coupled to the tray 90. The tray 90 may be referred to as a caddy, container, or by other descriptive terms in some embodiments. The medical devices of certain embodiments may be any implant or instrument used in accomplishing a medical procedure. The medical device may be, without limitation, similar to any of the medical devices described in association with FIGS. 1-3. The mechanisms 600 illustrated are respectively associated with the spinal surgical screws 10, at least by proximity. The mechanisms 600 of some embodiments include a distal portion 601 configured to couple with the tray 90. Each mechanism 600 illustrated is in a location on the tray 90 that is associated by proximity with a respective spinal surgical screw 10. An identifier 620 may be associated with one or more of the mechanisms 600. The identifier 620 may include a two dimensional bar code, RFID tag, or any device that is capable of retaining identifying information, including but not limited to, devices described in association with the identifier 120 herein.

In some embodiments, the multiple mechanisms 600 do not contact the respective multiple medical devices. Instead, respective medical devices may be associated with mechanisms 600 by proximity. In other embodiments, there may be contact and interdigitation between respective mechanisms 600 and medical devices, such as spinal surgical screws 10. In some embodiments, a mechanism 600 may not be releasable from the tray 90 unless the medical device is first removed.

FIG. 10 graphically illustrates a method of tracking medical devices delivered in a set that includes multiple medical devices. In the illustrated embodiment, a tray with two or more medical devices is provided. Identifiers associated with respective ones of the medical devices are also provided. The tray, medical devices, and identifiers may be the same as or similar to any of the items describe in association with FIGS. 1-9, or may be other items capable of practice of the method described.

Embodiments may also include giving instructions to detach one or more identifiers to record which medical devices have been used. An identifier may be removed from a portion of a tray or from a medical device, capsule, tag, or other component associated with respective medical devices. The instructions my be directed to a healthcare provider, a company sales representative, a computing device, or any other person or machine capable of effectively recording some or all of the information associated with the identifier and its associated medical implant or instrument. A healthcare provider may include physicians, nurses, technicians, hospitals, purchasing agents, governmental agencies, administrative staff, and others. Information on an identifier and that is associated with a medical device may also include a date of use, a time of use, a condition treated, a particular surgical procedure, a procedure type, a number of times sterilized, and other information that might be useful in tracking the safety, utility, and efficacy of a medical device. An identifier and medical device may also be associated in some embodiments with manufacturing information, such as but not limited to, material type, lot number, country where manufactured, manufacturing facility, time of manufacture, and manufacturing process employed.

The identifier, a part of the identifier, or information from the identifier may be physically or virtually associated with the chart or medical information of a patient. In some embodiments, instructions to specifically associate one or more medical devices with a patient who receives a medical device or with whom an instrument is used are given. Instructions may also be given to specifically associate one or more medical devices used with particular locations of use in particular patients. For example and with out limitation, the fact that a spinal surgical screw was implanted in the right pedicle of the L5 vertebra of a particular patient may be associated with an identifier. Consequently, if a corrective action such as a recall for the screw were initiated, a patient may only have to undergo replacement of the particular screw rather than all screws used in the procedure. Identifiers may be physically retained, sent to a specified party, or information from the identifiers may be collected, and the identifiers themselves discarded.

In some embodiments, detaching one or more identifiers is detectable after the identifiers have been detached. Detection may be by any effective means and includes, but is not limited to, the devices described in association with FIGS. 1-9. Detaching as used herein may include moving an identifier from a first position in a tray to a second position that indicates use of or tampering with a medical device.

Embodiments may also include giving instructions to return identifiers to a provider of the associated medical devices. A provider may be the original manufacturer or an agent or otherwise authorized party acting on behalf of the original manufacturer. For example, an agent could be a data processing or data collection organization, or a sales representative or employee of the original manufacturer.

Some embodiments of the invention further include checking the medical device, tray, or associated mechanism to detect whether a medical device has been removed from the mechanism or tray. If a medical device has been removed from the mechanism or tray, the medical device or tray may be returned to a manufacturing or processing facility to be identified properly, or scrapped if tracking has been lost for the device. If not, a method under the invention may include subsequently transferring a previously delivered and returned medical device to a previous potential user or to a new user. The term new user as used herein may also refer to a new potential user.

FIG. 11 illustrates another embodiment of the invention that is a method of tracking multiple medical devices. To accomplish the method, a tray configured to accept multiple medical devices and respective multiple identifiers is provided. The tray, medical devices, and identifiers may be the same as or similar to any of the items described in association with FIGS. 1-9, or may be other items capable of practicing the method described. The method also includes providing instructions to sterilize the medical devices carried in the tray. The instructions may be provided to any person, machine, or organization able to accomplish a sterilization procedure.

After sterilization, one or more of the medical devices may be used or consumed in a medical procedure. After the need for the medical devices provided has concluded, the tray with medical devices may be returned to a provider. The provider may be the original manufacturer or an agent or otherwise authorized party acting on behalf of the original manufacturer.

The provider or authorized party in another act of the embodiment receives the tray after one or more of the multiple medical devices have been removed. In some embodiments, one or more associated identifiers are retained in the tray for use by the provider or authorized party. In other embodiments, identifiers may be processed by another route. For example, information from identifiers may be stored in a file system or electronic database as associated with particular instruments or implants provided. Alternatively, or in addition, the physical identifiers may be retained by a user or at the direction of a user. In any circumstance, one or both of identifiers and medical devices may be associated with a patient in connection with whom the medical devices were used or may be associated with the user of the medical devices. A user may be a healthcare provider or related entity such as physicians, nurses, technicians, hospitals, purchasing agents, governmental agencies, administrative staff, and others.

Embodiments of the invention may also include recording the retained identifiers to at least track which medical devices have been used. The recorded information may be recorded and maintained by either or both of a user of the medical device and an original manufacturer or agent of either. Information may be passed between users, patients, and original manufactures under some embodiments.

In any of the embodiments of the present invention, the medical devices may include, be made of, treated, coated, filled, used in combination with, or have a hollow space or opening for containing artificial or naturally occurring materials and/or substances suitable for implantation in the human body. These materials, and/or substances, may include any source of osteogenesis, bone growth promoting materials, bone derived substances or products, demineralized bone matrix, mineralizing proteins, ossifying proteins, bone morphogenetic proteins, hydroxyapatite, genes coding for the production of bone, and bone including, but not limited to, cortical bone, antibiotics, cancer treating substances, infection treating substances, substances to therapeutically affect clotting or stenosis, or other disease treating substances. The medical devices can include, at least in part materials that are bioabsorbable and/or resorbable in the body.

While the invention has been described with reference to particular embodiments, it will be appreciated by those of ordinary skill in the art that various modifications can be made to the invention itself without departing from the spirit and scope thereof. All changes and modifications that are within the spirit of the invention are hereby anticipated and claimed.

What is claimed is:

1. A medical device tracking system comprising:
a tray;
a medical device carried by the tray; and
a mechanism coupled to the tray, the mechanism being associated with the medical device and comprising:
a distal portion configured to couple with the tray, and
an identifier associated with the mechanism;
wherein the mechanism captures the medical device relative to the tray and is configured such that removal of the medical device from at least a portion of the mechanism is thereafter detectable.

2. The system of claim 1 wherein the tray is a steam sterilizable tray configured to allow steam to enter the tray and contact the medical device carried by the tray.

3. The system of claim 1 wherein the medical device is a surgical screw.

4. The system of claim 1 wherein the medical device is a surgical instrument.

5. The system of claim 1 wherein the mechanism includes a structurally weakened portion to facilitate removal of the medical device.

6. The system of claim 1 wherein the mechanism is configured to at least in part be ruptured when the medical device is removed from the tray.

7. The system of claim 1 wherein the mechanism is configured to at least in part non-resiliently deform when the medical device is removed from the tray.

8. The system of claim 1 wherein the mechanism is configured to at least in part change color when the medical device is removed from the tray.

9. The system of claim 1 wherein the distal end snaps into the tray to couple with the tray.

10. The system of claim 1 further comprising a capsule in which the medical device is carried.

11. The system of claim 10 wherein the capsule includes an opening through which a sterilization substance is passed.

12. The system of claim 10 wherein the capsule is hermetically sealed.

13. The system of claim 12 wherein the medical device is a sterile medical device.

14. A medical device tracking system comprising:
a tray;
multiple medical devices carried by the tray; and
multiple mechanisms coupled to the tray, the mechanisms being respectively associated with the multiple medical devices, one or more of the mechanisms comprising:
a distal portion configured to couple with the tray in a location on the tray that is associated by proximity with the medical device, and
an identifier associated with the mechanism;
wherein the multiple mechanisms do not contact the respective multiple medical devices.

15. The system of claim 14 wherein one or more of the medical devices is a spinal implant.

16. The system of claim 14 wherein one or more of the medical devices is a surgical instrument.

17. A method of tracking medical devices delivered in a set that includes multiple medical devices comprising:
providing a tray;
providing two or more medical devices in the tray;
providing identifiers associated with respective ones of the medical devices, wherein detaching one or more identifiers from the tray or medical device is thereafter detectable;
giving instructions to detach one or more identifiers to record which medical devices have been used; and
detecting if one or more identifiers has been detached from the tray or medical device.

18. The method of claim 17 wherein the act of detaching one or more identifiers includes removing an identifier from the tray.

19. The method of claim 17 wherein the act of detaching one or more identifiers includes removing an identifier from the medical device.

20. The method of claim 17 further comprising giving instructions to specifically associate one or more medical devices used with particular patients.

21. The method of claim 17 further comprising giving instructions to specifically associate one or more medical devices used with particular locations of use in particular patients.

22. The method of claim 17 further comprising giving instructions to return identifiers to a provider of the associated medical devices.
